(19) United States
(12) Patent Application Publication
(10) Pub. No.: US 2026/0133208 A1
(43) Pub. Date: May 14, 2026

(54) DIAGNOSTIC DEVICE FOR DETECTING AND/OR IDENTIFYING A TARGET ANALYTE FROM A LIQUID SAMPLE

(71) Applicant: ACTIFAR, Mont-Saint-Guibert (BE)

(72) Inventors: Beatrice Maerevoet, Limal (BE); Olivier Naessens, Eghezée (BE)

(73) Assignee: ACTIFAR, Mont-Saint-Guibert (BE)

(21) Appl. No.: 18/944,444

(22) Filed: Nov. 12, 2024

(51) Int. Cl. G01N 33/76; B01L 3/00; G01N 21/77; G01N 21/78; G01N 33/543

(52) U.S. Cl. CPC G01N 33/76; B01L 3/5023; G01N 21/78; G01N 33/54388; B01L 2200/16; B01L 2300/069; B01L 2300/0825; G01N 2021/7759

(57) ABSTRACT

A diagnostic device for detecting and/or identifying a target analyte from a liquid sample is disclosed. The device includes: a support with an upper face and a lower face, and at least one reagent strip enclosed inside the support between the upper face and the lower face. The at least one reagent strip includes a test zone and reagents enabling the target analyte to be detected and/or identified. The upper face of the support includes: at least one opening designed to give access to a zone for depositing the liquid sample, the deposition zone being in fluid communication with the test zone of the at least one reagent strip, and a reading window opening onto the test zone of the at least one reagent strip where the target analyte is detected and/or identified, the support having at least one rupture zone designed to give a user access to a free end of the at least one reagent strip.

The present invention relates to a diagnostic device for detecting and/or identifying a target analyte from a liquid sample. In particular, the present invention relates to a pregnancy test based on the detection of the hCG hormone.

A device according to the present invention can enable the detection and/or identification of any type of analyte, often called the analyte of interest or target analyte. For example, a device according to the present invention can detect and/or identify an analyte of interest being a toxin, an organic compound, a protein, a peptide, a microorganism, a bacterium, a virus, an amino acid, a nucleic acid, a carbohydrate, a hormone, a steroid, a vitamin, a drug, a pollutant, a pesticide, an antigenic substance, a hapten or even an antibody.

Numerous diagnostic devices are available, some of which consist of a plastic shell (or support) containing a synthetic reagent strip (or detection tab) made of a plastic material, typically nitrocellulose. A more ecological approach aimed at considerably reducing the plastic waste generated by the use of such diagnostic devices has led to the development of diagnostic devices consisting of a paper or cardboard shell (or support) containing a reagent strip (or detection tab) made of a plastic material, typically nitrocellulose. The nitrocellulose membranes or strips are synthetic and non-biodegradable.

As for pregnancy tests, these devices are generally based on a biochemical immunoassay, testing for the presence of elevated levels of the human chorionic gonadotropin hormone (hCG) in the body fluids such as urine. The hormone hCG is generated at the start of the pregnancy and is therefore a very useful indicator analyte. Immunoassay is generally based on the use of anti-hCG antibodies and antigens adapted to bind to such antibodies at binding sites on the antibody.

Traditionally, pregnancy test devices are based on the use of a urine sample that infiltrates a reagent strip by capillary action. A reservoir of anti-hCG antibodies bound to a coloured particulate material is encountered first. If hCG is present, the antibodies bind to it, forming a complex which is carried along the reagent strip. Further along the reagent strip is a transverse linear zone containing an anchored reagent to which the hCG/antibody complex will bind. Also, if hCG is present in the urine, the hCG/antibody complex and associated coloured material will accumulate in this area, forming a coloured line visible on the reagent strip.

Unfortunately, the current diagnostic devices, such as current pregnancy tests, are not very environmentally friendly. In this sense, even if they have a paper or cardboard shell (or support), the current pregnancy tests are still waste that cannot be easily recycled, as they comprise a plastic reagent strip and a paper or cardboard shell (or support). After using such devices, the user is faced with a waste product comprising two materials that cannot be recycled/treated/disposed of in the same way, since plastic materials and materials such as paper or cardboard follow very different recycling, treatment or disposal routes.

To meet the expectations of consumers who are increasingly demanding environmentally-friendly products (green products) and/or to reduce environmental pollution and/or to meet regulatory obligations, there is a real need today to provide diagnostic devices that can be easily recycled, treated or disposed of.

To solve these problems, the invention provides a diagnostic device for detecting and/or identifying a target analyte from a liquid sample, comprising:
a support with an upper face and a lower face, and
at least one reagent strip enclosed inside said support between said upper face and said lower face, said at least one reagent strip comprising a test zone and reagents enabling said target analyte to be detected and/or identified,
   said upper face of said support comprising:
at least one opening designed to give access to a zone for depositing said liquid sample, said deposition zone being in fluid communication with said test zone of said at least one reagent strip, and
a reading window opening onto said test zone of said at least one reagent strip where said target analyte is detected and/or identified,
   said diagnostic device being characterised in that said support has at least one rupture zone designed to give a user access to a free end of said at least one reagent strip.

By the terms “said support has at least one rupture zone designed to give a user access to a free end of said at least one reagent strip”, it is understood within the meaning of the present invention that the support comprises one or more means enabling it to be broken and/or at least partially ruptured. In particular, according to the present invention, the “rupture zone” can allow the upper face of the support or the lower face of the support or both the upper face and the lower face of the support to be broken and/or ruptured, so as to give a user access to a free end of at least one reagent strip. Said support according to the present invention therefore has at least one rupture zone arranged to give a user access to a free end of at least one reagent strip.

By the terms “free end of said at least one reagent strip”, it is understood, within the meaning of the present invention, an end of at least one reagent strip which is not fixed to the support and which is directly accessible to a user following a rupture of the rupture zone, i.e. when the rupture zone has been used to break and/or rupture the support. According to the invention, the free end of at least one reagent strip is not fixed/attached/hooked to the support by any means. The free end is also not locked in the support in such a way that it can be easily grasped and separated from it.

With such a diagnostic device according to the invention having a rupture zone designed/arranged to give a user access to a free end of said at least one reagent strip, it is easy to separate at least one reagent strip (for example made of a plastic material) from the support (for example made of paper or cardboard). As a result, a user can easily direct at least one reagent strip made of a plastic material and the paper or cardboard support separately to the appropriate recycling/treatment/disposal channel. Among other things, this avoids incineration of the diagnostic device. A diagnostic device according to the invention is therefore environmentally friendly, meets consumer expectations in terms of reducing the ecological footprint and complies with current and future regulatory requirements. All this makes recycling and waste management possible, because each component can be treated individually in the appropriate way.

For the purposes of this invention, the term “sample” refers to anything that may contain an analyte for which analyte detection and/or identification is desired. The sample may be a biological sample, such as a biological fluid sample. Examples of biological fluids include urine, blood, plasma, serum, saliva, semen, stool, sputum, cerebrospinal fluid, tears, mucus, amniotic fluid or similar. Such samples may be human, animal or man-made.

For the purposes of this invention, the terms “fluid sample” or “liquid sample” refer to a material suspected of containing the analyte of interest, which material has sufficient fluidity to flow through a diagnostic device according to the invention.

The fluid sample or liquid sample can be used as obtained directly from the source or following pre-treatment so as to modify its character. Any pre-treatment of the sample may involve the preparation of plasma from blood, dilution of viscous fluids, filtration, distillation, separation, concentration, inactivation of interfering components and addition of reagents.

Typically, the sample is an aqueous solution or a biological fluid. The liquid sample can come from any source, such as physiological fluid, including blood, serum, plasma, saliva, sputum, eye lens solution, sweat, urine, milk, ascites fluid, mucus, synovial fluid, peritoneal fluid, transdermal exudates, pharyngeal exudates, bronchoalveolar lavage, tracheal aspirates, cerebrospinal fluid, semen, cervical mucus, vaginal or urethral secretions or amniotic fluid.

A solid material suspected of containing the analyte can be used as a sample after modification to form a liquid medium or to release the analyte.

As used herein, the term “biodegradable” refers to a material that is capable of being decomposed by bacteria or other living organisms, natural processes or other biological agents or means. For example, for the purposes of this invention, cardboard and paper can be considered biodegradable materials.

For the purposes of this invention, the term “fluid communication” refers to the positioning or arrangement of one or more materials such that the fluid is capable of flowing through the material or flowing between the materials by capillary, absorptive or axial flow. A material can be in “fluid communication” with another material regardless of the presence of fluid if it offers the ability to allow fluid flow between materials when fluid is present.

Within the meaning of the present invention, the terms “reagent strip” or “detection tab” refer to a part/component of the diagnostic device comprising reagents and a test zone in fluid communication with a deposit zone/sample reception zone.

For the purposes of this invention, the term “sample deposition area” (also referred to as “sample area”) refers to an area where a sample is brought into contact with the diagnostic device. According to one embodiment of the invention, the sample deposition zone is designed to receive a fluid sample (or liquid sample) which may contain an analyte of interest. In another embodiment, the sample deposition zone is immersed in a fluid sample (or liquid sample).

According to the invention, reagents capable of binding to the analyte of interest, in particular marker reagents capable of binding to the analyte of interest, can be positioned downstream of the sample deposition zone or can be positioned within the sample deposition zone. In addition, the test zone is arranged downstream of the sample zone and contains test and control zones or lines. The test zone contains a reagent or an adaptation that enables the analyte of interest to be retained in the test zone. In particular, the reagent or adapter included in the test zone comprises an immobilised capture reagent that binds to the analyte of interest. Therefore, as the liquid sample flows along the reagent strip, the analyte of interest first binds with a marker reagent and is then retained in the test zone.

Advantageously, according to the invention, said support is made of a biodegradable and/or recyclable material, such as paper or cardboard.

Preferably, the diagnostic device according to the invention is a single-use diagnostic device, which means that the diagnostic device is generally discarded after use.

Preferably, according to the invention, said at least one reagent strip is designed to detect an analyte comprising chorionic gonadotropin (hCG).

Advantageously, the diagnostic device according to the invention comprises a housing located on the lower face of said support and designed to accommodate at least one reagent strip. In particular, such a housing is designed to receive and hold in a stable position at least one reagent strip during the test, without it being fixed in the housing.

Preferably, according to the invention, said housing is arranged to accommodate said at least one reagent strip in order to hold it in place without fixing it to the support. Maintaining said at least one reagent strip in the housing in this way ensures a reliable diagnosis. The housing may, for example, be in the form of a cup/container in which said at least one reagent strip is immobilised but not fixed. For the purposes of the present invention, such a housing can simply be defined between the upper face and the lower face of the support, said at least one reagent strip being held between these two faces without being fixed to the support.

Preferably, according to the invention, said zone for depositing said liquid sample comprises an absorbent material in fluid communication with said at least one reagent strip.

According to one embodiment of the invention, said at least one reagent strip is made of a plastic material and/or a polymer such as nitrocellulose, for example.

Advantageously, according to the invention, said rupture zone is in the form of perforations or in the form of a predetermined point of weakness or in the form of a reversible fastening system or in the form of a pre-printed rupture line or in the form of a flexible hinge or in the form of an adhesive rupture zone or in the form of a release rupture zone or in the form of an assisted tear rupture zone.

Advantageously, the diagnostic device according to the invention is manufactured from materials compatible with safety and quality standards for medical devices.

Preferably, the diagnostic device according to the invention comprises a gripping zone or element located in a plane substantially parallel to the plane of the upper face or lower face of the support, said gripping zone being connected to the support by a connecting zone or element.

The present invention also relates to the use of a diagnostic device according to the invention for detecting and/or identifying a target analyte from a liquid sample, in particular for detecting and/or identifying chorionic gonadotropin (hCG).

Other characteristics, details and advantages of the invention will be apparent from the examples given below, by way of non-limitation and with reference to the attached figures.

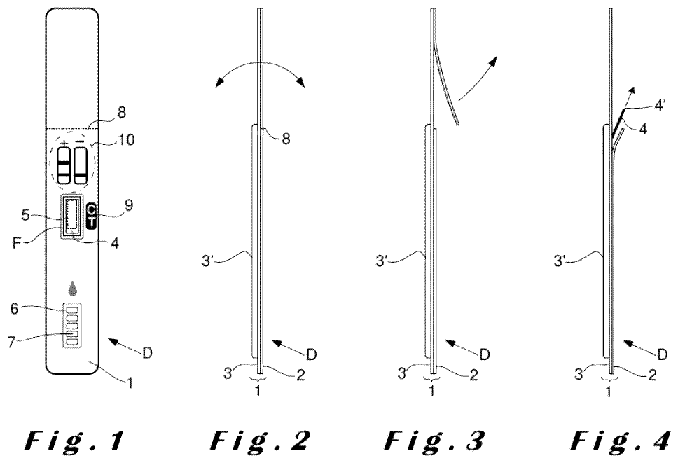

FIGS. 1 to 4 illustrate a first embodiment of a diagnostic device according to the invention.

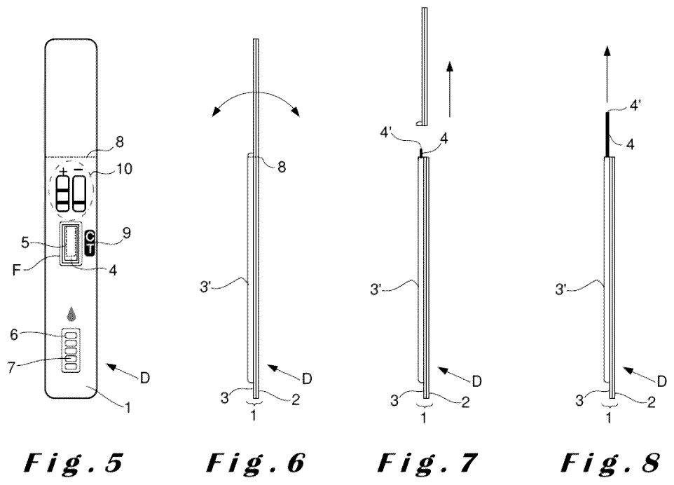

FIGS. 5 to 8 illustrate a second embodiment of a diagnostic device according to the invention.

FIG. 9 illustrates a further embodiment of a diagnostic device according to the invention.

### EXAMPLES

FIGS. 1 to 4 illustrate a first embodiment of a diagnostic device D according to the invention. According to this first embodiment, the diagnostic device D for detecting and/or identifying a target analyte from a liquid sample comprises a support 1 having an upper face 2 and a lower face 3 and a reagent strip 4 enclosed inside the support 1 between its upper face 2 and its lower face 3. According to this embodiment, the lower face 3 comprises a housing 3′designed to contain and hold the reagent strip 4 in a stable position, but without fixing it to the support 1 or to the housing 3′. The support 1 is made of a biodegradable material and has at least one rupture zone 8 designed to give a user access to a free end 4′of said reagent strip 4. The reagent strip 4 comprises a test zone 5 and reagents for detecting and/or identifying said target analyte. The upper face 2 of the support 1 comprises openings 6 arranged to give access to a zone 7 for depositing the liquid sample, the deposition zone 7 being in fluid communication with the test zone 5 of the reagent strip 4. The upper face 2 of the support 1 also includes a reading window F opening onto the test zone 5 of the reagent strip 4 where the detection and/or identification of the target analyte takes place. The diagnostic device as illustrated includes printouts 9, 10 enabling the user to interpret the results displayed in the reading window F.

After using the diagnostic device as described above and illustrated in FIGS. 1 to 4, the user can exert a movement on a part of the support 1 as indicated by the double arrow in FIG. 2. This movement will allow the fracture zone 8 to break so that part of the upper face 2 of the support 1 can be easily lifted as indicated by the arrow in FIG. 3. The user then has access to a free end 4′ of the reagent strip 4 contained in the housing 3′ and can easily extract the reagent strip 4 by grasping its free end 4′ as indicated by the arrow in FIG. 4 so as to separate the support 1 and the reagent strip 4.

FIGS. 5 to 8 illustrate a second embodiment of a diagnostic device according to the invention. The diagnostic device D for detecting and/or identifying a target analyte from a liquid sample according to this second embodiment comprises the same elements as those described for the first embodiment, only the rupture zone 8 being substantially in a different form.

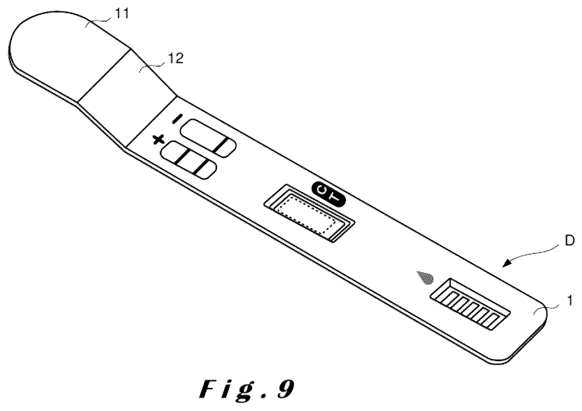

After using the diagnostic device D as described above and illustrated in FIGS. 5 to 8, the user can exert a movement on part of support 1 as indicated by the double arrow in FIG. 6. This movement will allow the rupture zone 8 to break in such a way that part of the upper face 2 of the support 1, part of the lower face 3 of the support 1 and part of the housing 3′ become detached from the rest of the diagnostic device D, for example by simple traction, as indicated by the arrow in FIG. 7. The user then has access to the free end 4′ of the reagent strip 4 and can easily extract the reagent strip 4 by grabbing/grasping its free end 4′ as indicated by the arrow in FIG. 8 so as to separate the support 1 and the reagent strip 4.

FIG. 9 illustrates a diagnostic device according to the invention. According to this embodiment, the diagnostic device comprises a gripping zone or element 11 located in a plane substantially parallel to the plane of the upper face 2 or lower face 3 of the support 1, the gripping zone 11 being connected to the support 1 by a connecting zone or element 12.

The present invention has been described above in connection with specific embodiments, which are illustrative and should not be considered limiting. Generally speaking, it will be obvious to the person skilled in the art that the present invention is not limited to the examples illustrated and/or described above.

The use of the verbs “comprise”, “include”, or any other variant, as well as their conjugations, can in no way exclude the presence of elements other than those mentioned.

The use of the indefinite article “a”, “an”, or the definite article “the”, to introduce an element does not exclude the presence of a plurality of these elements.

1. A diagnostic device for detecting and/or identifying a target analyte from a liquid sample, comprising:
a support with an upper face and a lower face, and
at least one reagent strip enclosed inside said support between said upper face and said lower face, said at least one reagent strip comprising a test zone and reagents enabling said target analyte to be detected and/or identified,
said upper face of said support comprising:
at least one opening designed to give access to a zone for depositing said liquid sample, said deposition zone being in fluid communication with said test zone of said at least one reagent strip, and
a reading window opening onto said test zone of said at least one reagent strip where said target analyte is detected and/or identified,
wherein said support has at least one rupture zone designed to give a user access to a free end of said at least one reagent strip.

2. The diagnostic device according to claim 1, said support is made of a biodegradable and/or recyclable material.

3. The diagnostic device according to claim 1, wherein the device is for single use only.

4. The diagnostic device according to claim 1, wherein said at least one reagent strip is designed to detect an analyte comprising chorionic gonadotropin (hCG).

5. The diagnostic device according to claim 1, comprising a housing located on the lower face of said support and arranged to accommodate said at least one reagent strip.

6. The diagnostic device according to claim 1, wherein said zone for depositing said liquid sample comprises an absorbent material in fluid communication with said at least one reagent strip.

7. The diagnostic device according to claim 1, wherein said at least one reagent strip is made of a plastic material and/or a polymer such as nitrocellulose, for example.

8. The diagnostic device according to claim 1, wherein said rupture zone is in the form of perforations or in the form of a predetermined point of weakness or in the form of a reversible fastening system or in the form of a pre-printed rupture line or in the form of a flexible hinge or in the form of an adhesive rupture zone or in the form of a release rupture zone or in the form of an assisted tear rupture zone.

9. The diagnostic device according to claim 1, wherein the device is manufactured from materials compatible with safety and quality standards for medical devices.

10. The diagnostic device according to claim 1, wherein the device comprises a gripping zone or element situated in a plane substantially parallel to the plane of the upper face or lower face of said support, said gripping zone being connected to the support by a connecting zone or element.

11. A method for detecting and/or identifying a target analyte from a liquid sample, wherein the target analyte comprises chorionic gonadotropin (hCG) and the method is performed with the diagnostic device of claim 1.
